(19) United States
(12) Patent Application Publication
(10) Pub. No.: US 2008/0171081 A1
(43) Pub. Date: Jul. 17, 2008

(54) PHARMACEUTICALLY ACCEPTABLE PHOSPHATE-GLYCEROL CARRYING BODIES

(76) Inventors: Anthony Ernest Bolton, Shannon (IE); Arkady Mandel, Mississauga (CA)

Correspondence Address: FOLEY & LARDNER LLP, 975 PAGE MILL ROAD, PALO ALTO, CA, 94304, US

(21) Appl. No.: 11/946,729

(22) Filed: Nov. 28, 2007

Related U.S. Application Data
(62) Division of application No. 10/348,601, filed on Jan. 21, 2003.
(60) Provisional application No. 60/421,116, filed on Jan. 22, 2002.
(60) Provisional application No. 60/351,427, filed on Jan. 28, 2002.
(60) Provisional application No. 60/364,620, filed on Mar. 18, 2002.
(60) Provisional application No. 60/372,106, filed on Apr. 15, 2002.
(60) Provisional application No. 60/400,857, filed on Aug. 2, 2002.

(30) Foreign Application Priority Data
Jan. 21, 2002 (CA) 2,368,656

(51) Int. Cl. A61K 9/127; A61P 29/00; A61P 37/00

(52) U.S. Cl. 424/450

(57) ABSTRACT

This invention relates to three-dimensional synthetic and semi-synthetic compositions having biological activity, and to the uses thereof in the treatment and/or prophylaxis of various disorders in mammalian patients. More particularly it relates to preparations and uses of synthetic and semi-synthetic bodies, such as liposomes, which after introduction into the body of a patient, produce beneficial anti-inflammatory, organ protective and immune regulatory effects. The invention also relates to treatments and compositions for alleviating inflammatory and autoimmune diseases and their symptoms.

### CROSS-REFERENCE TO RELATED APPLICATIONS

This application claims the benefit under 35 U.S.C. § 119(a) of Canadian Application No. 2,368,656, filed on Jan. 21, 2002, which application is herein incorporated by reference in its entirety.

This application further claims the benefit under 35 U.S.C. § 119(e) of the following applications: U.S. Provisional Application No. 60/______, which was converted pursuant to 37 C.F.R. § 1.53(c)(2)(i) from U.S. patent application Ser. No. 10/051,381, filed Jan. 22, 2002; U.S. Provisional Application No. 60/351,427, filed Jan. 28, 2002; U.S. Provisional Application No. 60/364,620, filed Mar. 18, 2002; U.S. Provisional Application 60/372,106, filed Apr. 15, 2002 and U.S. Provisional Application No. 60/400,857, filed Aug. 2, 2002, all of which applications are herein incorporated by reference in their entireties.

### BACKGROUND OF THE INVENTION

1. Field of the Invention

This invention relates to three-dimensional synthetic and semi-synthetic compositions having biological activity, and to the uses thereof in the treatment and/or prophylaxis of various disorders in mammalian patients. More particularly it relates to preparations and uses of synthetic and semi-synthetic bodies, which after introduction into the body of a patient, produce beneficial anti-inflammatory, organ protective and immune regulatory effects. The invention also relates to treatments and compositions for alleviating inflammatory and autoimmune diseases and their symptoms.

### REFERENCES

1. U.S. Pat. No. 4,485,054, issued Nov. 27, 1984, to Mezei et al.
2. U.S. Pat. No. 4,496,787, issued Jan. 29, 1985, to Touchais et al.
3. U.S. Pat. No. 4,812,314, issued Mar. 14, 1989 to Barenholz.
4. U.S. Pat. No. 4,938,763, issued Jul. 3, 1990 to Dunn et al.
5. U.S. Pat. No. 4,946,787, issued Aug. 7, 1990, to Eppstein et al.
6. U.S. Pat. No. 5,188,951, issued Feb. 23, 1993, to Tremblay et al.
7. U.S. Pat. No. 5,252,263, issued Oct. 12, 1993, to Hope et al.
8. U.S. Pat. No. 5,376,452, issued Dec. 27, 1994, to Hope et al.
9. U.S. Pat. No. 5,736,157, issued Apr. 7, 1998, to Williams.
10. U.S. Pat. No. 5,741,514, issued Apr. 21, 1998, to Barenholz et al.
11. U.S. Pat. No. 5,746,223, issued May 5, 1998, to Williams.
12. U.S. Pat. No. 5,843,474, issued Dec. 1, 1998, to Williams.
13. U.S. Pat. No. 5,858,400, issued Jan. 12, 1999, to Williams.
14. U.S. Pat. No. 6,297,870, issued Oct. 2, 2001, to Nanba.
15. U.S. Pat. No. 6,312,719, issued Nov. 6, 2001, to Hope et al.
16. International Publication No. WO 01/66785, published Sep. 13, 2001.
17. International Patent Application PCT/CA02/01398 to Vasogen Ireland Limited.
18. Lehniger, Biochemistry (1970)
19. Barenholz et al. “Liposomes as Pharmaceutical Dosage Forms”
20. New, R. C. “Liposomes: A Practical Approach”, IRL Press at Oxford University Press (1990).
21. Richard Harrigan—1992 University of British Columbia PhD Thesis “Transmembrane pH gradients in liposomes (microform): drug-vesicle interactions and proton flux”, published by National Library of Canada, (1993); University Microfilms order no. UM100406756; Canadian no. 942042220, ISBN 0315796936.
22. Griffin W S T et al. “Brain interleukin 1 and S-100 immunoreactivity are elevated in Down Syndrome and Alzheimer Disease.” Proceedings of the National Academy of Sciences USA. 86: 7611-7615 (1989).
23. Bliss, T. V. P., et al. “A synaptic model of memory: long-term potentiation in the hippocampus.” Nature. 361: 31-39 (1993).
24. Murray, C. A., et al. “Evidence that increase hippocampal expression of the cytokine interleukin-1B is a common trigger for age and stress-induced impairments in long-term potentiation.” J. Neuroscience. 18: 2974-2981 (1998).
25. Mogi, M., et al. “Interleukin (IL)-1 beta, IL-1, IL-4, IL-6 and transforming growth factor-alpha levels are elevated in ventricular cerebrospinal fluid in juvenile parkinsonism and Parkinson's Disease.” Neuroscience Letters. 211: 13-16 (1996).
26. Giannessi D, Del Ry S, Vitale R L “The role of endothelins and their receptors in heart failure.” Pharmacol Res 2001 February 43:2 111-26.
27. Van de Stolpe A, Van der Saag P T, “Intercellular adhesion molecule-1” J. Mol. Med. (1996) 74:112-33.

All of the above publications, patents and patent applications are herein incorporated by reference in their entirety to the same extent as is if each individual publication, patent or patent application was specifically and individually indicated to be incorporated by reference in its entirety.

2. Background of the Invention

Professional antigen-presenting cells (APCs), including dendritic cells (DCs) and macrophages (Mph), actively capture and process antigens (Ags), clear cell debris, and remove infectious organisms and dying cells, including the residues from dying cells. During this process, APCs can stimulate the production of either inflammatory Th1 pro-inflammatory cytokines (IL-12, IL-1, INF-γ, TNF-α, etc.); or regulatory Th2/Th3 cytokines (such as IL-10, TGF-β, IL-4 etc.) dominated responses; depending on the nature of the antigen (Ag) or phagocytosed material and the level of APC maturation/activation.

APCs remove cellular debris, some of which is derived from cell membranes of the body, some from bacterial and parasitic infections and commensal organisms, such as gut bacteria. While some of this cellular debris will initiate a pro-inflammatory response, some initiates a protective and anti-inflammatory response.

A normally functioning immune system is capable of distinguishing between the antigens of foreign invading organisms (non-self) and tissues or debris derived from “self,” mounting an immune response only against foreign antigens. When a patient's immune system fails to discriminate between self and non-self, autoimmune disorders arise.

### SUMMARY OF THE INVENTION

This invention is directed to the discovery that pharmaceutically acceptable bodies, such as liposomes, beads or similar particles, which comprise phosphate-glycerol groups, will, upon administration to a mammalian patient, cause an anti-inflammatory effect and therefore may be used to treat a number of diseases. These bodies may further comprise as a minor component an inactive constituent, and/or constituent which is active through a different mechanism.

In a preferred embodiment, the invention is directed to a composition of matter capable of producing an anti-inflammatory response in vivo in a mammal, said composition comprising pharmaceutically acceptable bodies of a size from about 20 nanometers (nm) to 500 micrometers (μm), comprising a plurality of phosphate-glycerol groups or groups convertible to such groups. Preferably, the bodies are essentially free of non-lipid pharmaceutically active entities. Preferably the phosphate-glycerol groups constitute 60%-100% of the active groups on the bodies. Following administration to a mammal, the bodies, through the phosphate-glycerol groups, are believed to interact with the immune system. As a result, when so administered an anti-inflammatory response is elicited.

In another embodiment, this invention is directed to a three-dimensional synthetic or semi-synthetic body, otherwise referred to herein as pharmaceutically acceptable bodies, having a size ranging from 20 nm to 500 μm, and having been modified to comprise, as a major component, at least one anti-inflammatory promoting ligand wherein said ligand has phosphate-glycerol groups.

In still another embodiment, this invention is directed to three-dimensional synthetic and semi-synthetic bodies, otherwise referred to herein as pharmaceutically acceptable bodies, having sizes ranging from 20 nm to 500 μm, and having phosphate-glycerol groups on the surface thereof.

In another aspect, the invention is directed to a method for treating a T-cell function-mediated disorder comprising administering to a mammalian patient an effective amount of pharmaceutically acceptable bodies carrying an effective number of phosphate-glycerol groups to inhibit and/or reduce the progression of the T-cell function-mediated disorder.

This invention is further directed to a method for treating an inflammatory disorder comprising administering to a patient an effective amount of pharmaceutically acceptable bodies carrying an effective number of phosphate-glycerol groups to inhibit and/or reduce the progression of the inflammatory disorder.

Yet another embodiment of this invention is a method for treating an endothelial function disorder comprising administering to a mammalian patient an effective amount of pharmaceutically acceptable bodies carrying an effective number of phosphate-glycerol groups to inhibit and/or reduce the progression of the endothelial function disorder.

Another embodiment is a method for treating an immune disorder characterized by inappropriate cytokine expression comprising administering to a mammalian patient an effective amount of pharmaceutically acceptable bodies carrying an effective number of phosphate-glycerol groups to inhibit and/or reduce the progression of the immune disorder.

This invention is further directed to a process for treating or prophylaxis of a mammalian cardiac disorder, the presence of or the susceptibility to which is detectable by observing a prolonged QT-c interval on an electrocardiogram of the patient, which process comprises administering to a mammalian patient suffering therefrom or susceptible thereto a pharmaceutical composition comprising pharmaceutically acceptable biocompatible synthetic or semi-synthetic bodies, otherwise referred to herein as pharmaceutically acceptable bodies, and a pharmaceutically acceptable carrier, wherein at least a portion of said bodies have a size in the range from about 20 nm to 500 μm, and wherein the surfaces of said bodies have been modified to carry, as a major component, at least one anti-inflammatory promoting group, said group being a phosphate-glycerol.

Another embodiment of the invention is a pharmaceutical composition, in unit-dosage form, for administration to a mammalian patient, comprising pharmaceutically acceptable bodies and a pharmaceutically acceptable carrier, wherein at least a portion of the bodies has a size in the range from about 20 nm to 500 μm, and wherein the surfaces of said bodies comprise phosphate-glycerol groups or groups convertible to phosphate-glycerol groups, said unit dosage comprising from about 500 to about 2.5×10⁹ bodies.

A further embodiment of this invention is a pharmaceutical composition comprising a pharmaceutically acceptable biocompatible synthetic or semi-synthetic bodies (otherwise referred to herein as pharmaceutically acceptable bodies) and a pharmaceutically acceptable carrier, wherein at least a portion of said bodies has a size from about 20 nm to 500 μm, and wherein the surfaces of said bodies have been modified to comprise, as a major component, at least one anti-inflammatory promoting group, wherein said group is phosphate-glycerol.

A still further embodiment of this invention is a pharmaceutical composition comprising pharmaceutically acceptable biocompatible synthetic or semi-synthetic bodies (otherwise referred to herein as pharmaceutically acceptable bodies) and a pharmaceutically acceptable carrier, wherein at least a portion of said bodies has a from about 20 nm to 500 μm, and comprises cardiolipin.

Optionally, the bodies described above may additionally comprise an inactive constituent surface group and/or a constituent surface group, which is active through another mechanism, e.g. phosphatidylserine. (See, e.g. Fadok et al., International Publication WO 01/66785).

In another embodiment, this invention is directed to lyophilized or freeze-dried pharmaceutically acceptable bodies carrying phosphate-glycerol groups or groups convertible to phosphate-glycerol groups, and kits comprising lyophilized or freeze dried bodies comprising phosphate-glycerol groups, or groups convertible to phosphate-glycerol groups, and a pharmaceutically acceptable carrier.

In another aspect, this invention is directed to a method for treating a T-cell function-mediated disorder comprising administering to a mammalian patient suffering from or at risk of suffering from a T-cell function mediated disorder, an effective amount of a composition comprising pharmaceutically acceptable bodies having a size from about 20 nm to about 500 μm, comprising on the surface thereof a plurality of phosphate-glycerol groups, or groups convertible to said phosphate-glycerol groups, such that upon administration, the progression of the T-cell function mediated disorder is inhibited and/or reduced.

Yet another embodiment of this invention is directed to a method for treating an endothelial function disorder comprising administering to a mammalian patient suffering from or at risk of suffering from an endothelial function disorder an effective amount of a composition comprising pharmaceutically acceptable bodies having a size of from about 20 nm to about 500 μm, comprising on the surface thereof a plurality of phosphate-glycerol groups, or groups convertible to said phosphate-glycerol groups, such that upon administration, the progression of the endothelial function disorder is inhibited and/or reduced.

Another embodiment of this invention is directed to a method for treating an immune disorder in a mammalian patient suffering from or at risk of suffering from an immune disorder, comprising administering to said mammalian patient an effective amount of a composition comprising pharmaceutically acceptable bodies having a size of from about 20 nm to about 500 μm, comprising on the surface thereof a plurality of phosphate-glycerol groups, or groups convertible to said phosphate-glycerol groups, such that upon administration, the progression of the immune disorder is inhibited and/or reduced.

Another embodiment of this invention is directed to a method for treating an inflammatory disorder in a mammalian patient suffering from or at risk of suffering from an inflammatory disorder, comprising administering to said mammalian patient an effective amount of a composition comprising pharmaceutically acceptable bodies having a size of from about 20 nm to about 500 μm, comprising on the surface thereof a plurality of phosphate-glycerol groups, or groups convertible to said phosphate-glycerol groups, such that upon administration, the progression of the inflammatory disorder is inhibited and/or reduced.

The present invention can also be viewed, from another aspect, as the use of a receptor on cells on the mammalian immune system, e.g. macrophages, which specifically bind to the phosphate-glycerol group. The invention embraces bodies comprising ligands and groups that will bind to such receptor and consequently produce an anti-inflammatory response. Accordingly, the present invention can be defined as bodies comprising ligands or active groups thereof that compete with the binding or uptake of phosphate-glycerol expressing bodies as described herein by antigen-presenting cells. A person skilled in the art can readily determine whether a particular body is one which will so compete, by conducting a simple experiments. For example, the bodies can be tested with a readily available monocytic cell line such as U937 cells. In a first experiment, U937 cells are incubated with fluorescent labeled PG liposomes alone, and in other experiments the U937 cells are incubated in the presence of both fluorescent labeled PG liposomes and differing amounts of test compound. If the uptake of the fluorescent labeled PG liposomes in the other experiments is reduced in comparison with that of the first experiment, then the test compound is competing for the specific receptor and is a compound within the scope of the present invention.

### BRIEF DESCRIPTION OF THE DRAWINGS

FIG. 1 is a bar graph presentation of the results of Example 1 below, murine contact hypersensitivity (CHS, acute T-cell mediated inflammatory model) experiments using liposomes in accordance with a preferred embodiment of the invention, in comparison with other liposomes and controls.

FIG. 2 is a similar graphical presentation, showing the use of liposomes of various phosphatidylglycerol (PG) contents, in the murine CHS model, Example 2 below.

FIG. 3 is a similar graphical presentation of the results of Example 3 below where different concentrations of 75% PG liposomes were used in the murine CHS model.

FIG. 4 is a similar graphical presentation of the results of Example 4 below, where different concentrations of 100% PG liposomes were used in the murine CHS model.

FIG. 5 is a similar graphical presentation of the results of Example 5 below, using liposomes of different sizes in the CHS model.

FIG. 6 is a similar graphical presentation of the results of Example 6 below, using a murine model of delayed type hypersensitivity (DHS, chronic T-cell mediated inflammatory model).

FIG. 7 is a similar graphical presentation of the results of Example 7 below, cardiolipin liposomes in a DHS murine model.

FIG. 8 is a similar graphical presentation of the results of Example 8 below, cardiolipin liposomes in a CHS murine model.

FIG. 9 shows the change in the percentage of excitatory post-synaptic potential (EPSP) slope in control and treated mice, which is indicative of the effect on long term potentiation (LTP), Example 9.

FIG. 10 displays the data shown in FIG. 9 in the format of a bar chart, Example 9 below.

FIG. 11 Shows the difference in the concentration of the anti-inflammatory cytokine IL-4 in the hippocampus of control and treated animals, Example 10 below.

FIG. 12 shows the difference in the concentration of the pro-inflammatory cytokine IL-1β in a single cell suspension of spleen cells of control and treated animals, Example 11 below.

FIG. 13 shows the difference in the concentration of TNF-α in the U937 monocyte cell line treated with varying concentration of 75% PG liposomes, Example 12 below.

FIG. 14 is a graphical presentation of the results of Example 13 below, endothelin-1 content in ears of mice treated according to a preferred embodiment of the invention versus control.

FIG. 15 is a graphical presentation of the results of Example 14, ICAM-1 positive cells from HUVEC cultures in the presence and absence of compositions of the preferred embodiment of the invention.

### DESCRIPTION OF PREFERRED EMBODIMENTS

According to the present invention, pharmaceutically acceptable bodies carrying phosphate-glycerol groups on their surface are administered to patients. Without being limited to any theory, is believed that these bodies interact with the immune system of the patient with accompanying beneficial effects such as inhibition of pro-inflammatory cytokines in vivo and/or promotion of anti-inflammatory cytokines. The reacting cells may be immune cells such as professional or non-professional antigen presenting cells, endothelial cells, regulatory cells such as NK-T cells and others.

These pharmaceutically acceptable bodies include synthetic and semi-synthetic bodies having shapes which are typically but not exclusively spheroidal, cylindrical, ellipsoidal, including oblate and prolate spheroidal, serpentine, reniform etc., and sizes from about 20 nm to about 500 μm in diameter, preferably measured along its longest axis, and comprising phosphate-glycerol groups on the surface thereof.

The pharmaceutically acceptable bodies have phosphate-glycerol groups of predetermined characteristics on the exterior surface. Without being limited to any theory, it is believed that these groups are capable of interacting with the appropriate receptor(s), other than exclusively the PS receptor, on antigen presenting cells in vivo. The structure of these groups may be synthetically altered and include all, part of or a modified version of the original phosphate-glycerol group. For example, the negatively charged oxygen of the phosphate group of the phosphate-glycerol group may be converted to a phosphate ester group (e.g., L-OP(O)(OR′)(OR″), where L is the remainder of the phosphate-glycerol group, R is —CH₂CH(OH)CH₂OH and R″ is alkyl of from 1 to 4 carbon atoms or hydroxyl substituted alkyl of from 2 to 4 carbon atoms, and 1 to 3 hydroxyl groups provided that R″ is more readily hydrolyzed in vivo than the R′ group; to a diphosphate group including diphosphate esters (e.g., L-OP(O)(OR′)OP(O)(OR″)₂ wherein L and R′ are as defined above and each R″ is independently hydrogen, alkyl of from 1 to 4 carbon atoms, or a hydroxyl substituted alkyl of from 2 to 4 carbon atoms and 1 to 3 hydroxyl groups provided that the second phosphate group [-P(O)(OR″)₂] is more readily hydrolyzed in vivo than the R′ group; or to a triphosphate group including triphosphate esters (e.g., L-OP(O)(OR′)OP(O)(OR″)OP(O)(OR″)₂ wherein L and R′ are defined as above and each R″ is independently hydrogen, alkyl of from 1 to 4 carbon atoms, or a hydroxyl substituted alkyl of from 2 to 4 carbon atoms and 1 to 3 hydroxyl groups provided that the second and third phosphate groups are more readily hydrolyzed in vivo than the R′ group; and the like. Such synthetically altered phosphate-glycerol groups are capable of expressing phosphate-glycerol in vivo and, accordingly, such altered groups are phosphate-glycerol convertible groups.

Phosphatidylglycerol is a known compound. It can be produced, for example, by treating the naturally occurring dimeric form of phosphatidylglycerol, cardiolipin, with phospholipase D. It can also be prepared by enzymatic synthesis from phosphatidylcholine using phospholipase D—see, for example, U.S. Pat. No. 5,188,951 Tremblay, et al. Chemically, it has a phosphate-glycerol group and a pair of similar but different C₁₈-C₂₀ fatty acid chains.

As used herein the term “PG” is intended to cover phospholipids carrying a phosphate-glycerol group with a wide range of at least one fatty acid chains provided that the resulting PG entity can participate as a structural component of a liposome. Preferably, such PG compounds can be represented by the Formula I:

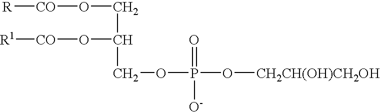

where R and R¹ are independently selected from C₁-C₂₄ hydrocarbon chains, saturated or unsaturated, straight chain or containing a limited amount of branching wherein at least one chain has from 10 to 24 carbon atoms. Essentially, the lipid chains R and R¹ form the structural component of the liposomes, rather than the active component. Accordingly, these can be varied to include two or one such lipid chains, the same or different, provided they fulfill the structural function. Preferably, the lipid chains may be from about 10 to about 24 carbon atoms in length, saturated, mono-unsaturated or polyunsaturated, straight-chain or with a limited amount of branching. Laurate (C12), myristate (C14), palmitate (C16), stearate (C18), arachidate (C20), behenate (C22) and lignocerate (C24) are examples of useful saturated lipid chains for the PG for use in the present invention. Palmitoleate (C16), oleate (C18) are examples of suitable mono-unsaturated lipid chains. Linoleate (C18), linolenate (C18) and arichidonate (C20) are examples of suitable poly-unsaturated lipid chains for use in PG in the liposomes of the present invention. Phospholipids with a single such lipid chain, also useful in the present invention, are known as lysophospholipids. The present invention also extends to cover use of liposomes in which the active component is the dimeric form of PG, namely cardiolipin but other dimers of Formula I are also suitable. Preferably, such dimers are not synthetically cross-linked with a synthetic cross-linking agent, such as maleimide but rather are cross-linked by removal of a glycerol unit as described by Lehniger, Biochemistry, p. 525 (1970) and depicted in the reaction below:

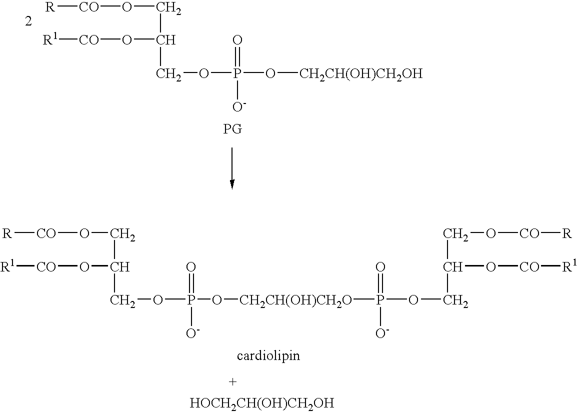

where each R and R¹ are independently as defined above.

As noted above and again without being limited to any theory, the PG group and its dimer are believed to be a ligand since it is believed that it binds to a specific site on a protein or other molecule (“PG receptor”) and, accordingly, this molecule of phosphatidylglycerol (and its dimeric form) is sometimes referred to herein as a “ligand” or a “binding group.” Such binding is believed to take place through the phosphate-glycerol group —O—P(═O)(OH)—O—CH₂—CH(OH)—CH₂—OH, which is sometimes referred to herein as the “head group,” “active group,” or “binding group.” In view of the above, reference to “binding,” “binding group,” or “ligand” herein is not to infer any mechanism or mode of action. Nevertheless, it is believed that the above phosphate-glycerol groups are presented on the exterior surfaces of the bodies of the present invention for interaction with components of the patient's immune system. This interaction, it should be noted, is not the same as the specific interaction of apoptotic cells with the phosphatidylserine receptor on antigen presenting cells.

The term “phosphate-choline” refers to the group —O—P(═O)(OH)—O—CH₂—CH₂—N⁺(CH₃)₃, which is attached to the remainder of the lipid as shown in the following structure:

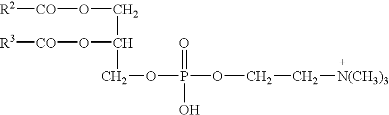

and salts thereof, wherein R² and R³ are independently selected from C₁-C₂₄ hydrocarbon chains, saturated or unsaturated, straight chain or containing a limited amount of branching wherein at least one chain has from 10-24 carbon atoms.

Examples of “three-dimensional body portions” or pharmaceutically acceptable bodies” include biocompatible synthetic or semi-synthetic entities such as liposomes, solid beads, hollow beads, filled beads, particles, granules and microspheres of biocompatible materials, natural or synthetic, as commonly used in the pharmaceutical industry. The beads may be solid or hollow, or filled with biocompatible material. The term “biocompatible” refers to substances which in the amount employed are either non-toxic or have acceptable toxicity profiles such that their use in vivo is acceptable. Likewise the term “pharmaceutically acceptable” as used in relation to “pharmaceutically acceptable bodies” refers to bodies comprised of one or more materials which are pharmaceutically acceptable and suitable for delivery in vivo. Such bodies can include liposomes formed of lipids, one of which is PG. Alternatively, the pharmaceutically acceptable bodies can be solid beads, hollow beads, filled beads, particles, granules and microspheres of biocompatible materials, which comprise one or one or more biocompatible materials such as polyethylene glycol, poly(methylmethacrylate), polyvinylpyrrolidone, polystyrene and a wide range of other natural, semi-synthetic and synthetic materials, with phosphate-glycerol groups attached thereto.

As noted above, analogues of phosphatidylglycerol with modified active groups, which also interact with PG receptors on the antigen presenting cells, through the same receptor pathway as PG or otherwise resulting in an anti-inflammatory reaction in the recipient body are contemplated within the scope of the term phosphatidylglycerol. This includes, without limitation, compounds in which one or more of the hydroxyl groups and/or the phosphate group is derivatized, or in the form of a salt. Many such compounds form free hydroxyl groups in vivo, upon or subsequent to administration and, accordingly, comprise convertible PG groups.

Preferred compositions of matter are liposomes, which may be composed of a variety of lipids. Preferably, however, none of the lipids are positively charged. In the case of liposomes, phosphatidyl glycerol PG may constitute the major portion or the entire portion of the liposome layer(s) or wall(s), oriented so that the phosphate-glycerol group portion thereof is presented exteriorly, to act as the binding group, and the lipid chain or chains form the structural wall.

Liposomes, or lipid vesicles, are sealed sacs, in the micron or sub-micron range, the walls (monolayer or multilayer) of which comprise suitable amphiphiles. They normally contain an aqueous medium, although for the present invention the interior contents are unimportant, and generally inactive. Accordingly, in a preferred embodiment, the liposomes, as well as other pharmaceutically acceptable bodies, are essentially free of non-lipid pharmaceutically active entities (e.g. <1%) and more preferably are free of non-lipid pharmaceutically acceptable entities. Such liposomes are prepared and treated so that the active groups are presented exteriorly on the liposomal body. The PG in the liposomes of the preferred embodiments of this invention thus serves as both a ligand and a structural component of the liposome itself.

Thus a preferred embodiment of this invention provides liposomal bodies which expose or can be treated or induced to expose, on their surfaces, one or more phosphate-glycerol groups to act as binding groups. Phosphatidylglycerol is a preferred PG ligand and such lipids should comprise from 10%-100% of the liposome, with the balance being an inactive constituent, e.g. phosphatidylcholine PC, or one which acts through a different mechanism, e.g. phosphatidylserine PS, or mixtures of such. Inactive co-constituents such as PC are preferred.

As used herein, the term “PS” is intended to cover phosphatidylserine and analogues/derivatives thereof provided that such analogues/derivatives enhance or stimulate the activity of the phosphatidylserine receptor.

At least 10% by weight of such liposome is composed of PG, preferably at least 50%, more preferably from 60-100% and most preferably from 70-90%, with the single most preferred embodiment being about 75% by weight of PG.

Mixtures of PG liposomes with inactive liposomes and/or with liposomes of phospholipids acting through a different mechanism can also be used, provided that the total amount of PG remains above the minimum of about 10% and preferably above 60% in the total mixture.

As regards to non-liposomal bodies for use in the present invention, these as noted to include biocompatible solid or hollow beads of appropriate size. The biocompatible non-liposomal synthetic or semi-synthetic bodies may be selected from polyethylene glycol, poly(methylmethacrylate), polyvinylpyrrolidone, polystyrene and a wide range of other natural, semi-synthetic and synthetic materials, with phosphate-glycerol groups attached to the surfaces thereof. Such materials include biodegradable polymers, such as disclosed by Dunn, et al. U.S. Pat. No. 4,938,763, which is hereby incorporated by reference in its entirety.

Biodegradable polymers are disclosed in the art and include, for example, linear-chain polymers such as polylactides, polyglycolides, polycaprolactones, polyanhydrides, polyamides, polyurethanes, polyesteramides, polyorthoesters, polydioxanones, polyacetals, polyketals, polycarbonates, polyorthocarbonates, polyphosphazenes, polyhydroxybutyrates, polyhydroxyvalerates, polyalkylene oxalates, polyalkylene succinates, poly(malic acid), poly(amino acids), polyvinylpyrrolidone, polyethylene glycol, polyhydroxycellulose, chitin, chitosan, and copolymers, terpolymers and combinations thereof. Other biodegradable polymers include, for example, gelatin, collagen, etc.

Suitable substances for derivatization to attach the phospholipid(s), or portions thereof with groups or binding groups, to three-dimensional bodies are commercially available e.g. from Polysciences Inc., 400 Valley Road, Warrington, Pa. 18976, or from Sigma Aldrich Fine Chemicals. Methods for their derivatization are known in the art. Specific preferred examples of such methods are disclosed in International Patent Application PCT/CA02/01398 Vasogen Ireland Limited, which is incorporated herein by reference.

It is contemplated that the patient may be a mammal, including but not limited to humans and domestic animals such as cows, horses, pigs, dogs, cats and the like.

Phospholipids are amphiphilic molecules (i.e. amphiphiles), meaning that the compound comprises molecules having a polar water-soluble group attached to a water-insoluble hydrocarbon chain. The amphiphiles serving as the layers of the matrix have defined polar and apolar regions. The amphiphiles can include, in addition to PG in this invention, other, naturally occurring lipids used alone with the phospholipid carrying the active group, or in a mixture with another. The amphiphiles serving as the layer(s) of the liposomes can be inert, structure-conferring synthetic compounds such as polyoxyethylene alkylethers, polyoxyethylene alkylesters and saccharosediesters.

Methods of preparing liposomes of the appropriate size are known in the art and do not form part of this invention. Reference may be made to various textbooks and literature articles on the subject, for example, the review article “Liposomes as Pharmaceutical Dosage Forms”, by Yechezkel Barenholz and Daan J. A. Chrommelin, and literature cited therein, for example New, R. C. “Liposomes: A Practical Approach”, IRL Press at Oxford University Press (1990).

The diameter of the liposomes, as well as the other pharmaceutically acceptable bodies, of the preferred embodiment of this invention is from about 20 nm to about 500 μm, more preferably from about 20 nm to about 1000 nm, more preferably from about 50 nm to about 500 nm, and most preferably from about 80 nm to about 120 nm (preferably measured along its longest axis). In one embodiment, the diameter of the liposome is from 60 nm to 500 μm.

The pharmaceutically acceptable bodies may be suspended in a pharmaceutically acceptable carrier, such as physiological sterile saline, sterile water, pyrogen-free water, isotonic sterile saline, and phosphate buffer sterile solutions (e.g. sterile aqueous solutions comprising phosphate buffer), as well as other non-toxic compatible substances used in pharmaceutical formulations, such as, for example, adjuvants, buffers, preservatives, and the like. Preferably, the pharmaceutically acceptable bodies are constituted into a liquid suspension in a sterile biocompatible liquid such as buffered sterile saline and administered to the patient by any appropriate route which exposes it to one or more components of the immune system, such as intra-arterially, intravenously or most preferably intramuscularly or subcutaneously.

It is contemplated that the pharmaceutically acceptable bodies may be freeze-dried or lyophilized so that they may be later resuspended for administration. This invention is also directed to a kit of part comprising lyophilized or freeze-dried binding group-carrying bodies and a pharmaceutically acceptable carrier, such as physiological sterile saline, sterile water, pyrogen-free water, isotonic saline, and phosphate buffer solutions (e.g. sterile aqueous solutions comprising phosphate buffer), as well as other non-toxic compatible substances used in pharmaceutical formulations, such as, for example, adjuvants, buffers, preservatives, and the like. Protectants for freeze drying, as known in the art, for example lactose or sucrose, may also be included.

A preferred manner of administering the pharmaceutically acceptable bodies to the patient is a course of injections, administered daily, several times per week, weekly or monthly to the patient, over a period ranging from a week to several months. The frequency and duration of the course of the administration is likely to vary from patient to patient, and according to the condition being treated, its severity, and whether the treatment is intended as prophylactic, therapeutic or curative. Its design and optimization is well within the skill of the attending physician. Intramuscular injection, especially via the gluteal muscle, is most preferred. One particular injection schedule, in at least some of the indications of the invention, is an injection, via the gluteal muscle, of an appropriate amount of bodies on day 1, a further injection on day 2, a further injection on day 14, and then “booster” injections at monthly intervals, if appropriate.

It is postulated that, in many embodiments of the present invention, pharmaceutically acceptable bodies comprising the PG groups as binding groups on their surface are acting as modifiers of the patient's immune system, in a manner similar to that of a vaccine. Accordingly they are used in quantities and by administration methods to provide a sufficient localized concentration of the bodies at the site of introduction. Quantities of such bodies appropriate for immune system modification may not be directly correlated with body size of a recipient and can, therefore, be clearly distinguished from drug dosages, which are designed to provide therapeutic levels of active substances in a patient's bloodstream and tissues. Drug dosages are accordingly likely to be much larger than immune system modifying dosages.

The correlation between weights of liposomes and numbers of liposomes is derivable from the knowledge, accepted by persons skilled in the art of liposomal formulations, that a 100 nm diameter bilayer vesicle has 81,230 lipid molecules per vesicle, distributed approximately 50:50 between the layers (see Harrigan—1992 University of British Columbia PhD Thesis “Transmembrane pH gradients in liposomes (microform): drug-vesicle interactions and proton flux”, published by National Library of Canada, Ottawa, Canada (1993); University Microfilms order no. UMI00406756; Canada no. 942042220, ISBN 0315796936). From this one can calculate, for example, that a dose of 5×10⁸ vesicles, of the order of the dose used in the specific in vivo examples below, is equivalent to 4.06×10¹³ lipid molecules. Using Avogadro's number for the number of molecules of lipid in a gram molecule (mole), 6.023×10²³, one determines that this represents 6.74×10⁻¹¹ moles which, at a molecular weight of 729 for PG is approximately 3.83×10⁻⁸ gm, or 38.3 ng of PG for such dosage.

The quantities of the pharmaceutically acceptable bodies to be administered will vary depending on the nature of the mammalian disorder it is intended to treat and on the identity and characteristics of the patient. Preferably, the effective amount of pharmaceutically acceptable bodies is non-toxic to the patient, and is not so large as to overwhelm the immune system. When using intra-arterial, intravenous, subcutaneous or intramuscular administration of a sterile aqueous suspension of pharmaceutically acceptable bodies, it is preferred to administer, for each dose, from about 0.1-50 ml of liquid, containing an amount of bodies generally equivalent to 10%-1000% of the number of leukocytes normally found in an equivalent volume of whole blood. Preferably, the number of bodies administered per delivery to a human patient is in the range from about 500 to about 2.5×10⁹ (<250 ng of bodies, in the case of liposomes, pro-rated for density differences for other embodiments of bodies), more preferably from about 1,000 to about 1,500,000,000, even more preferably 10,000 to about 100,000,000, and most preferably from about 200,000 to about 2,000,000.

Since the pharmaceutically acceptable bodies are acting, in the process of the invention, as immune system modifiers, in the nature of a vaccine, the number of such bodies administered to an injection site for each administration maybe a more meaningful quantitation than the number or weight of bodies per unit of patient body weight. For the same reason, it is now contemplated that effective amounts or numbers of bodies for small animal use may not directly translate into effective amounts for larger mammals (i.e. greater than 5 kg) on a weight ratio basis.

The present invention is indicated for use in prophylaxis and/or treatment of a wide variety of mammalian disorders where T-cell function, inflammation, endothelial dysfunction and inappropriate cytokine expression are involved. A patient having or suspected of having such a disorder may be selected for treatment. “Treatment” refers to a reduction of symptoms, such as, but not limited to, a decrease in the severity or number of symptoms of the particular disease or a limit on the further progression of symptoms.

With respect to T-cell function (T-cell mediated) disorders, these disorders include any and all disorders mediated at least in party by T-cells and include for example, ulcers, wounds, and autoimmune disorders including, but not limited to diabetes, scleroderma, psoriasis and rheumatoid arthritis.

The invention is indicated for use with inflammatory allergic reactions, organ and cell transplantation reaction disorders, and microbial infections giving rise to inflammatory reactions. It is also indicated for use in prophylaxis against oxidative stress and/or ischemia reperfusion injury, ingestion of poisons, exposure to toxic chemicals, radiation damage, and exposure to airborne and water-borne irritant substances, etc., which cause damaging inflammation. It is also indicated for inflammatory, allergic and T-cell-mediated disorders of internal organs such as kidney, liver, heart, etc.

With respect to disorders involving inappropriate cytokine expression for which the present invention is indicated, these include any and all disorders involving inappropriate cytokine expression and include, for example, neurodegenerative diseases. Neurodegenerative diseases, including Down's syndrome, Alzheimer's disease and Parkinson's disease, are associated with increased levels of certain cytokines, including interleukin-1β (IL-1β) (see Griffin W S T et al. (1989); Mogi M. et al. (1996)). It has also been shown that IL-1β inhibits long-term potentiation in the hippocampus (Murray, C. A. et al. (1998)). Long-term potentiation in the hippocampus is a form of synaptic plasticity and is generally considered to be an appropriate model for memory and learning (Bliss, T. V. P. et al. (1993)). Thus, inappropriate cytokine expression in the brain is currently believed to be involved in the development and progression of neurodegenerative diseases and neuroinflammatory disorders.

Thus, the invention is indicated for the treatment and prophylaxis of a wide variety of mammalian neurodegenerative and other neurological disorders, including Downs syndrome, Alzheimer's disease, Parkinson's disease, senile dementia, depression, Huntingdon's disease, peripheral neuropathies, Guillain Barr syndrome, spinal cord diseases, neuropathic joint diseases, chronic inflammatory demyelinating disease, neuropathies including mononeuropathy, polyneuropathy, symmetrical distal sensory neuropathy, neuromuscular junction disorders, myasthenias and amyotrophic lateral sclerosis (ALS). Treatment and prophylaxis of these neurodegenerative diseases represents a particularly preferred embodiment of the invention, with treatment of Alzheimer's disease, Parkinson's disease and ALS particularly preferred.

Regarding disorders involving endothelial dysfunction, the present invention is indicated for the treatment and prophylaxis of a wide variety of such mammalian disorders including, any and all disorders mediated at least in part by endothelial dysfunction and include, for example, cardiovascular diseases, such as atherosclerosis, peripheral arterial or arterial occlusive disease, congestive heart failure, cerebrovascular disease (stroke), myocardial infarction, angina, hypertension, etc., vasospastic disorders such as Raynaud's disease, cardiac syndrome X, migraine etc., and the damage resulting from ischemia (ischemic injury or ischemia-reperfusion injury). In summary, it can be substantially any disorder the pathology of which involves an inappropriately functioning endothelium.

Further indications for the compositions and processes of the present invention include the treatment of patients to accelerate their rate of wound healing and ulcer healing, and treatment of patients prior to surgical operations, to accelerate their rate of recovery from surgery including their rate of healing of surgical wounds and incisions.

In regard to “cardiac disorders,” the present invention is indicated for the treatment and prophylaxis of a wide variety of such mammalian disorders including, any and all disorders relating to the heart and include, for example, ventricular arrhythmias (ventricular tachycardia or fibrillation) and sudden death from heart disease. Susceptibility of patients to cardiac disorders such as arrhythmias and sudden cardiac death is often indicated by prolonged QT-c intervals in the heart beat rhythm. Administration of compositions according to the preferred embodiments of the invention is believed to reduce QT-c intervals in mammalian patients, indicative of reduced susceptibility of to arrhythmia and sudden cardiac death.

The invention is further described, for illustrative purposes, in the following non-limiting examples.

### EXAMPLES

In the examples below, the following abbreviations have the following meanings. If an abbreviation is not defined, it has it generally acceptable meaning.
μg=microgram
μL=microliter
μm=micrometer
μM=micromolar
CHS contact hypersensitivity
cm=centimeter
DMSO=dimethylsulfoxide
DNFB=2,4-dinitrofluorobenzene
DHS=delayed-type hypersensitivity
EtOH=ethanol
g=gram
hrs=hours
Hz=hertz
IM=intramuscular
IP=intraperitoneal
kg=kilogram
LPS=lipopolysaccharide
LTP=long−term potentiation
mg=milligram
min=minutes
ml=milliliter
mM=millimolar
ms=millisecond
ng=nanogram
nm=nanometer
nM=nanomolar
PBS=phosphate-buffered saline
PCR=polymerase chain reaction
POPS=1-palmitoyl-2-oleoyl-sn-glycero-3-[phospho-L-serine], referred to in the examples herein as PS
POPG=1-palmitoyl-2-oleoyl-sn-glycero-3-[phospho-rac-(1-glycerol)]], referred to in the examples herein as PG
POPC=1-palmitoyl-2-oleoyl-sn-glycero-3-phosphocholine, referred to in the examples herein as PC
RPM=revolutions per minute
S=second

Unless otherwise stated, the precise form of the lipids used in the experiments was POPS, POPG and POPC as set out above.

### Example 1

Liposomes of 100±20 nm in average diameter were prepared according to standard methods known in the art and had the following compositions:

Group A—100% PS

Group B—100% PG

Group C—control, no liposomes.

A stock suspension of each liposome composition containing 4.8×10¹⁴ liposomes per ml was diluted with PBS to give an injection suspension containing 6×10⁶ particles per ml. The liposomal suspensions were injected into female BALB/c mice (Jackson Laboratories) aged 6-8 weeks and weighing 19-23 g, to determine the effect on ear swelling in the murine contact hypersensitivity (CHS) model. The CHS model tests for Th1-mediated inflammatory reactions.

The animals were assigned to one of 3 groups, with 5 animals in each group. Groups A and B received approximately 3×10⁵ of the above-identified liposomes (i.e., 100% PC and 100% PG, respectively), in a volume of approximately 50 μl. Group C was a control group, receiving no liposomes.

### Protocol

The following experiments were performed:

TABLE I

| | | | | | | | | Day 7 |
|---|---|---|---|---|---|---|---|---|
| | Lipo- | | | | | | | (24 |
| Group | somes | Day 1 | Day 2 | Day 3 | Day 4 | Day 5 | Day 6 | hours) |
| A | 100% | Injected | Injected | Injected | Injected | Injected | Injected | Ear |
| | PS | then | | | | | then | measured |
| | | sensitized | | | | | challenged | |
| B | 100% | Injected | Injected | Injected | Injected | Injected | Injected | Ear |
| | PG | then | | | | | then | measured |
| | | sensitized | | | | | challenged | |

On Days 1-6, mice of Groups A and B were injected with the respective liposomes preparations. Approximately 300,000 liposomes were injected in 501 volume via intramuscular (IN) injection, for a total administration over the test period of about 1,800,000 liposomes. Mice of the control group (Group C) received no liposomes, but were sensitized, challenged and tested in the same way as Groups A and B, as described below.

### Sensitization

On Day 1, following liposome injection for that day, mice were anaesthetized with 0.2 ml 5 mg/ml sodium pentobarbital via IP injection. The abdominal skin of the mouse was sprayed with 70% EtOH and a scalpel blade was used to remove about a one-inch diameter patch of hair from the abdomen. The shaved area was then painted with 25 μl of 0.5% 2,4-dinitrofluorobenzene (DNFB) in 4:1 acetone: olive oil using a pipette tip.

### Challenge

Following liposome injection on day 6, mice were challenged with DNFB by painting 10 μl of 0.2% DNFB on the dorsal surface of the right ear with a pipette tip and by painting 10 μl of vehicle on the left ear with a pipette tip.

### Results

On Day 7, 24 hours after challenge, each animal was anaesthetized with Halothane, and ear thickness was measured using a Peacock spring-loaded micrometer. Data was expressed as the difference between the treated right ear thickness and the thickness of the vehicle-treated left ear. The experiments were repeated three times, on similar animals. Increase in ear swelling was used as a measure of CHS response. The significance of the data was determined by the two-tailed student's t-test. A P value of <0.05 was considered significant.

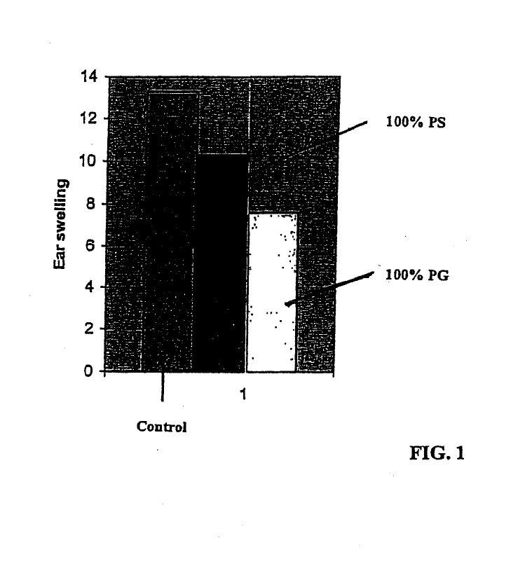

The results are presented in FIG. 1, a bar graph showing the mean values from the three experiments of ear swelling, reported in μm.

FIG. 1 shows that a significant reduction in ear swelling was achieved by injection of liposomes according to the present invention. The reduction achieved with 100% PG liposomes is substantially greater than that from 100% PS liposomes.

### Example 2

Liposomes of 100±20 nm in average diameter were prepared according to standard methods known in the art and had the following compositions:

Group A—100% PG

Group B—75% PG, 25% PC

Group C—50% PG, 50% PC

Group D—25% PG, 75% PC

Group E—PBS only

Group F—no injection

A stock suspension of each liposome containing 4.8×10¹⁴ liposomes per ml was diluted to give an injection suspension containing 12×10⁶ liposomes per ml. The liposomal suspensions were used to inject into mice to determine the effect on ear swelling in the murine CHS model, a biological system useful for assaying Th1-mediated inflammatory reactions. For these experiments, female BALB/c mice (Jackson Laboratories) aged 6-8 weeks and weighing 19-23 g were used.

The animals were assigned to one of 6 groups (Groups A-F, above) with 10 animals in each group. Control groups were also included that received no injections (Group F) or injections of PBS with no liposomes (Group E). Animals in Groups A-D were injected with 50 μl of the above-identified liposome suspensions, each containing about 6×10⁵ liposomes.

### Protocol

The test involves sensitization (Sens) with a potentially inflammation-causing substance, injection of liposomes (Inj) in test animals or PBS in controls and challenge (Chal) with the potentially inflammation-causing substance following measurement (Meas) to determine whether the injection of liposomes are effective against the development of inflammation by the challenge.

The following experiments were performed:

| | Lipo- | Day | Day | Day | Day | Day | Day | Day |
|---|---|---|---|---|---|---|---|---|
| Group | somes | 1 | 2 | 3 | 4 | 5 | 6 | 7 |
| A | 100% PG | Sens & | Inj | Inj | Inj | Inj | Chal & | Meas |
| | | Inj | | | | | Inj | |
| B | 75% PG | Sens & | Inj | Inj | Inj | Inj | Chal & | Meas |
| | | Inj | | | | | Inj | |
| C | 50% PG | Sens & | Inj | Inj | Inj | Inj | Chal & | Meas |
| | | Inj | | | | | Inj | |
| D | 25% PG | Sens & | Inj | Inj | Inj | Inj | Chal & | Meas |
| | | Inj | | | | | Inj | |
| E | None | Sens & | Inj | Inj | Inj | Inj | Chal & | Meas |
| | (PBS | Inj | | | | | Inj | |
| | only) | | | | | | | |
| F | none | Sens | | | | | Chal | Meas |

On days 1-6 the mice were injected with the respective liposomes as indicated above. Liposomes were injected in 50 μl volume via IM injection, i.e., 600,000 liposomes per injection, for a total administration over the test period of 3,600,000 liposomes. Mice of the control group received no liposomes but were sensitized, challenged and tested in the same way as the other groups of mice, as described below.

### Sensitization (Sens)

On Day 1, following liposome injection for that day, mice were anaesthetized with 0.2 ml 5 mg/ml sodium phenobarbital via IP injection. The abdominal skin of the mouse was sprayed with 70% EtOH and a blade was used to remove about a one inch diameter of hair from the abdomen. The bare area was painted with 25 μl of 0.5% 2,4-dinitrofluorobenzene (DNFB) in 4:1 acetone:olive oil using a pipette tip.

### Challenge (Chal)

On Day 6, following liposomes injection for that day, mice were challenged (Chal) with DNFB as follows: 10 μl of 0.2% DNFB was painted on the dorsal surface of the right ear with a pipette tip and 10 μl of vehicle was painted on the left ear with a pipette tip.

### Results

On Day 7, 24 hours after challenge, each animal was anaesthetized with Halothane, and ear thickness was measured (Meas) using a Peacock spring-loaded micrometer. Increase in ear swelling was used as a measure of CHS response. Data was expressed as the difference in the treated right ear thickness minus the thickness of the vehicle treated left ear. The significance between the two groups is determined by a two-tailed student's t-test. A P value of <0.05 is considered significant.

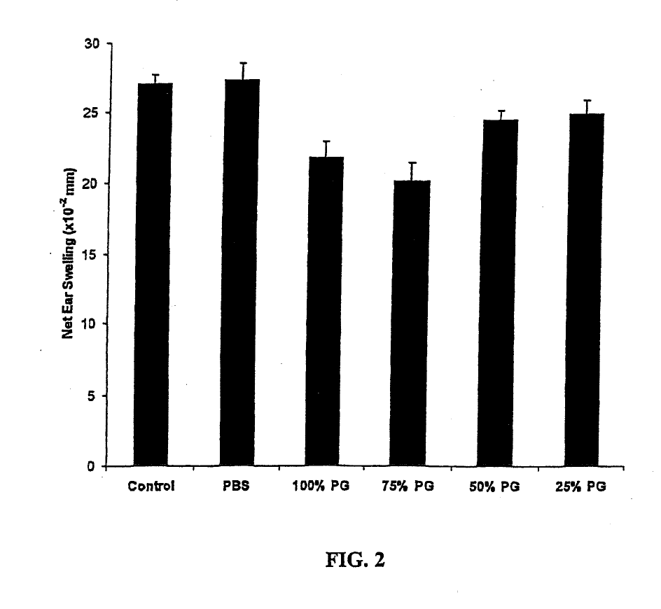

The results are presented graphically in FIG. 2, a bar graph showing ear swelling in μm. The mean value from the respective experiments was used in compiling the graph.

FIG. 2 shows that a significant reduction in ear swelling with both 100 and 75% PG is achieved, showing that both these concentrations protect against the development of inflammation resulting from contact with the allergenic substance, DNFB. The 50% and the 25% PG liposomes also showed reductions as compared with both controls, but the differences did not reach statistical significance in this experiment.

### Example 3

Liposomes of 100±20 nm in average diameter were prepared according to standard methods known in the art and were composed of 75% PG, 25% PC. A stock suspension containing 4.8×10¹⁴ liposomes per ml was used as before and diluted in PBS to give an injection suspension containing the following concentrations of liposomes:

| | | | Liposomes | |
|---|---|---|---|---|
| | | Concentration | per | Animals in |
| Group | Liposomes | (liposomes per mL) | injection | Group |
| A | 75% PG, 25% | 12 × 10¹¹ | 6 × 10¹⁰ | 10 |
| | PC | | | |
| B | 75% PG, 25% | 12 × 10⁹ | 6 × 10⁸ | 10 |
| | PC | | | |
| C | 75% PG, 25% | 12 × 10⁸ | 6 × 10⁷ | 16 |
| | PC | | | |
| D | 75% PG, 25% | 12 × 10⁷ | 6 × 10⁶ | 16 |
| | PC | | | |
| E | 75% PG, 25% | 12 × 10⁶ | 6 × 10⁵ | 16 |
| | PC | | | |
| F | none (PBS | | | 16 |
| | only) | | | |

BALB-c mice were divided into six groups (Groups A-F) including a control group receiving no liposomes but injected with 50 μL of PBS (Group F). Mice were sensitized on the flank, injected with their selected liposomal dose, intramuscularly to the right leg muscle, on the same day as, but after, sensitisation (day 1) and on days 2, 3, 4, and 5. On day 6 they were both injected and challenged on the ear as described in Example 1. The thickness of the ear was measured as described 24 hours after the challenge.

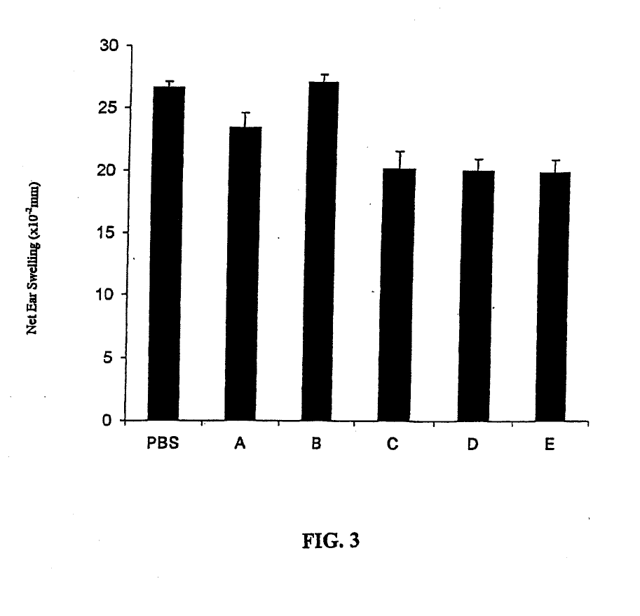

The results (FIG. 3) show a significant difference between the control group (Group F) and Group C (12×10⁸ liposomes per ml) and between the control group and Group D (12×10⁷ liposomes per ml) and between the control group and Group E (12×10⁶ liposomes per ml). There was little difference between the control group and Groups A or B (12×10¹¹ and 12×10⁹ liposomes per ml, respectively), suggesting that there is an optimum range of liposome concentrations above which the beneficial effects may be reduced. In other experiments, a decrease in effect was also be observed as the concentration of the liposomes was decreased below 12×10⁴ liposomes per ml.

### Example 4

Liposomes of formulation 100% PG and 100±20 nm in average size were prepared according to standard methods. Four groups (Groups A-D) of 10 mice were sensitised, injected and challenged in accordance with the procedure and schedule described in Example 3, with the following numbers of 100% PG liposomes delivered in a 50 μl suspension.

Group A—6×10⁷

Group B—6×10⁶

Group C—6×10⁵

Group D—6×10⁴

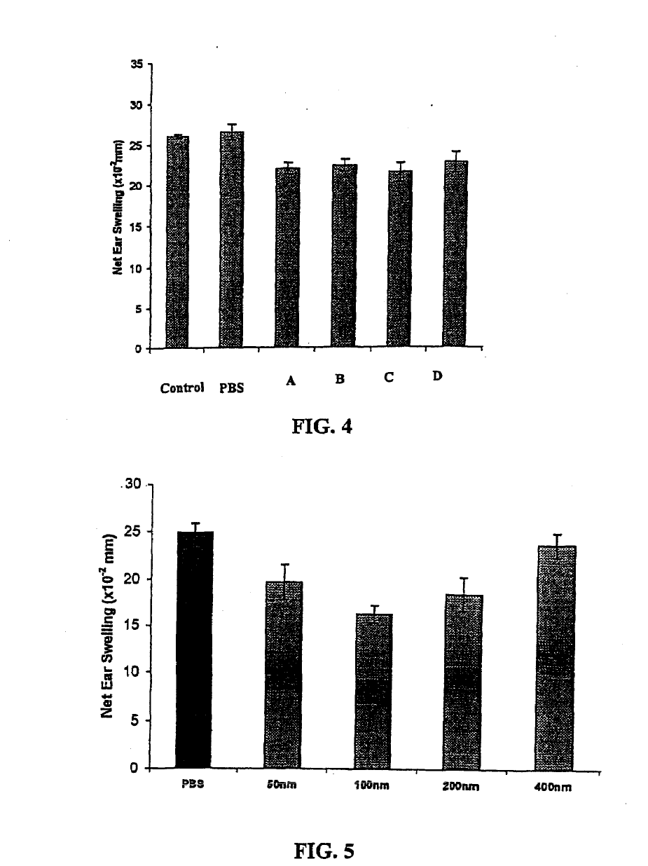

The results, along with the PBS control from Example 4, are presented in similar bar graph form in FIG. 4. A significant reduction in ear swelling, as compared with the control group is to be noted for each of the test groups, but with little difference between the various groups.

### Example 5

Liposomes of composition 75% PG, 25% PC and of 50, 100, 200, of 400 nm in average diameter were prepared by standard methods. They were tested the murine CHS model, as in Examples 3 and 4, using 6×10⁵ liposomes in 50 μl suspensions for each injection, and a sensitisation-injection-challenge schedule and procedure as in Example 3. The groups were as follows:

| | |
|---|---|
| Group A | 50 nm liposomes |
| Group B | 100 nm liposomes |
| Group C | 200 nm liposomes |
| Group D | 400 nm liposomes |
| Group E | no liposomes |

The results are presented in FIG. 5. The result from Group D, using the 400 nm diameter liposomes, is not significantly different from the control group (Group E), indicating a probable size range criticality in this model.

### Example 6

A stock suspension of 75% PG liposomes of 100±20 nm in average diameter containing 4.8×10¹⁴ liposomes per ml was diluted to give an injection suspension containing 6×10⁵ liposomes per ml. The liposomal suspensions were used to inject into mice, to determine the effect on ear swelling in the murine DHS model. As in Example 1, female BALB/c mice (Jackson Laboratories) aged 6-8 weeks and weighing 19-23 g were used.

The animals were assigned to one of 3 groups with 10 animals in each group. A control group (Group C) received only PBS injections. Animals of Groups A and B were injected with 50 μl of a suspension containing 6×10⁵ liposomes.

### Protocol

On days 13-18 the mice were injected with the 75% PG liposomes as indicated below. Liposomes were injected in 50 μl volume via IM injection, i.e., 600,000 liposomes per injection, for a total administration over the test period of 3,600,000 liposomes. Sensitization and challenge took place as described in Example 2.

| DAY | TREATMENT |
|---|---|
| 1 | Sensitized |
| 6 | Challenged |
| 7 | Measured |
| 12 | Challenged |
| 13 | Measured & Injected |
| 14 | Injected |
| 15 | Injected |
| 16 | Measured & Injected |
| 17 | Injected |
| 18 | Injected & Challenged |
| 19 | Measured |

### Results

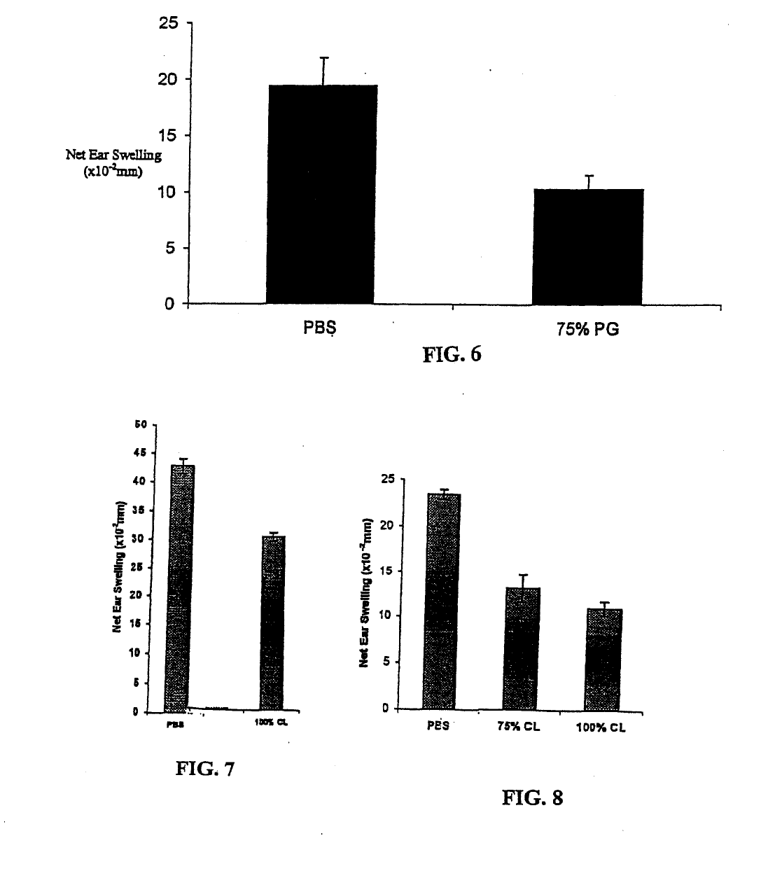

The results are presented graphically in accompanying FIG. 6 and show that 75% PG is effective in the DHS model on day 16, 24 hours after the third injection following the second challenge.

### Example 7

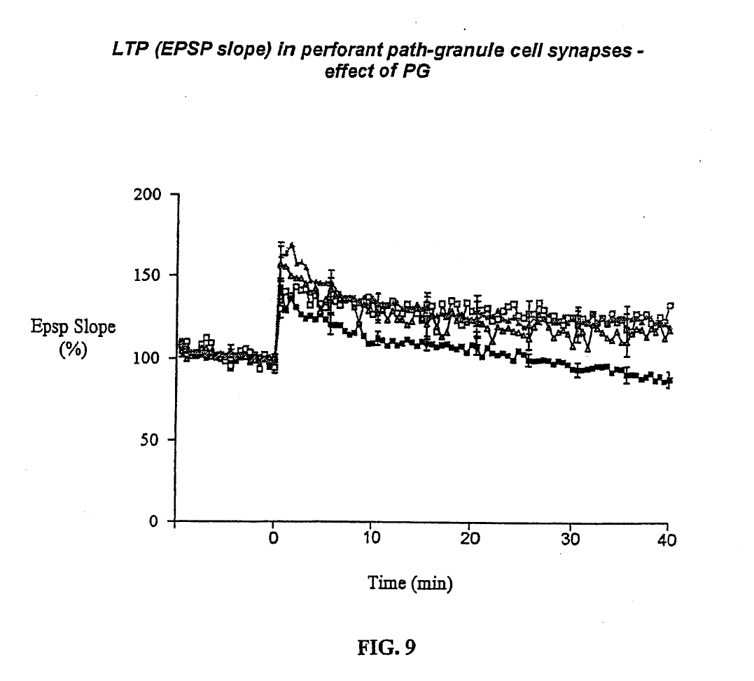

Liposomes of composed of 100% cardiolipin (CL) and 100±20 nm in average diameter were prepared, by standard methods. These were used at a dosage of 6×10⁵ liposomes per 50 μl per injection in the murine DHS model described in Example 6. Data obtained from animals injected with CL liposomes (Group A; 10 animals) was compared to data obtained from animals receiving only PBS (Group B; 10 animals). The sensitisation, injection and challenge procedures were as described in Example 2. The ear thickness measurement results, taken on day 19, 24 hrs after the 6^{th} injection, are presented in FIG. 7. The results showed a significant reduction in ear swelling within the CL-injected test (Group A).

### Example 8

Liposomes of 100 nm in average diameter, and comprising either 100% cardiolipin or 75% cardiolipin and 25% PC, were prepared by standard methods. Three groups (Groups A-C) of 10 mice were sensitised on day 1. A control group received injections of PBS on days 1, 2 and 6 (Group C). The other two groups received injections, of 6×10⁵ 100% cardiolipin liposomes (Group A) or of 6×10⁵ 75% cardiolipin liposomes (Group B), liposomes in 50 μl per injection according to the same schedule. The mice were challenged on day 7, and the ear thickness measured, as described in the previous examples.

FIG. 8 shows the mean measurements in each group. Both groups receiving CL liposomes showed a statistically significant suppression of CHS compared to the control group

### Example 9

To study the cellular and molecular mechanisms underlying cognitive function, the Long-Term Potentiation (LTP) animal model is used. LTP is a form of synaptic plasticity that occurs in the hippocampal formation, which has been proposed as a biological substrate for learning and memory (Bliss et al. Nature 361:31-39 (1990)). LTP in rats is monitored electrophysiologically by methods well known to those in the art. The animals are then sacrificed to investigate biochemical changes in hippocampal tissues. Comparing the results of electrophysiological data with biochemical hippocampal changes is useful for determining how the cellular events that underlie LTP may be altered in animals suffering from diseases or disorders associated with neuroinflammation such as aging, stress, Alzheimer's disease, and bacterial infection.

Systemic administration of lipopolysaccharide (LPS), a cell-wall component of Gram-negative bacteria, provokes an activation of the immune system by inducing an increase in pro-inflammatory cytokines such as IL-1. As noted above, one example of a neuronal deficit induced by LPS and IL-1: is the impairment of LTP in the hippocampus. An indicator of LTP is the mean slope of the population excitatory post-synaptic potential (epsp). Upon tetanic stimulation, the epsp slope (%) increases sharply indicating increased synaptic activity. LPS-induced inhibition of LTP reduces the increase in slope, and/or causes the epsp slope to revert more rapidly to base line, indicating that the increased synaptic activity is short-lived. Accordingly measurements of the epsp slope (%) at timed intervals after tetanic stimulation can be used to reflect memory and the loss thereof following an inflammatory stimulus as well as inflammation in the hippocampus of the brain.

Liposomes of 100±20 nm in average diameter were prepared as according to standard methods known in the art and were composed of 75% PG and 25% PC. A stock suspension of the liposomes containing about 2.9×10¹⁴ liposomes per ml was diluted with PBS to give an injection suspension containing about 1.2×10⁷ liposomes per ml. This was then used to inject into rats, to determine the effect on LPS-induced impairment of LTP. For these experiments, male Wistar rats (BioResources Unit, Trinity College, Dublin), weighing approximately 300 g, were used.

The animals were assigned to one of four groups, 8 animals in each group to be treated as follows:

| | |
|---|---|
| Group A | saline + control |
| Group B | saline + PG |
| Group C | LPS + control |
| Group D | LPS + PG |

150 μl of each above-identified preparation was injected via IM injection on days 1, 13, and 14. Groups B and D received a total of 5,400,000 liposomes (1,800,000 liposomes per injection). The LTP procedure and tissue preparation procedure were carried out on day 0.

### LTP Procedure

Rats were anaesthetized by IP injection of urethane (1.5 g/kg). Rats received either LPS (100 μg/kg) or saline intraperitoneally. Three hours later a bipolar stimulating electrode and a unipolar recording electrode were placed in the perforant path and in the dorsal cell body region of the dentate gyrus respectively. Test shocks of 0.033 Hz were given and responses recorded for 10 min before and 45 min after high frequency stimulation (3 trains of stimuli delivered at 30 s intervals, 250 Hz for 200 ms).

Rats were killed by decapitation. The hippocampus, the tetanized and untetanized dentate gyri, the cortex and entorhinal cortex were dissected on ice, sectioned and frozen in 1 ml of Krebs solution (composition of Krebs in mM: NaCl 136, KCl 2.54, KH₂PO₄ 1.18, MgSO₄.7H₂O 1.18, NaHCO₃ 16, glucose 10, CaCl₂ 1.13) containing 10% DMSO.

### Results

The results are shown in FIG. 9. The graph shows the difference in the excitatory post-synaptic potential (epsp) recorded in cell bodies of the granule cells. The data presented are means of seven to eight observations in each treatment group and are expressed as mean percentage change in epsp slope every 30 s normalized with respect to the mean value in the 5 minutes immediately prior to tetanic stimulation. FIG. 9 shows that the LPS-induced inhibition of LTP in perforant path-granule cell synapses was overcome by pre-treatment with the PG liposomes. The filled triangles represent Group A (saline+control), the open triangles represent Group B (saline+PG), the filled squares represent Group C (LPS+control) and the open squares represent Group D (LPS+PG).

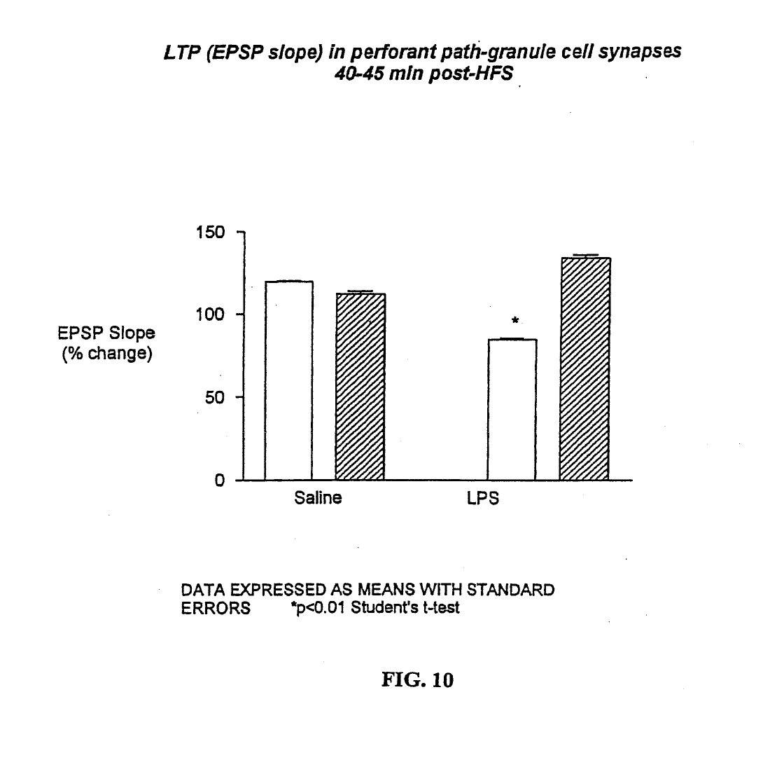

FIG. 10 shows that analysis of the mean values 40-45 minutes post tetanic stimulation indicate that the population epsp slope was decreased in the control-LPS group (open bars) and that the PG liposomes (hashed bars) significantly reversed this effect (*p<0.01). As an index of memory and learning functionality, the improvement in sustainability of LTP demonstrated in this Example indicates suitability of the treatment for dementias e.g. Alzheimer's disease and memory impairment.

### Example 10

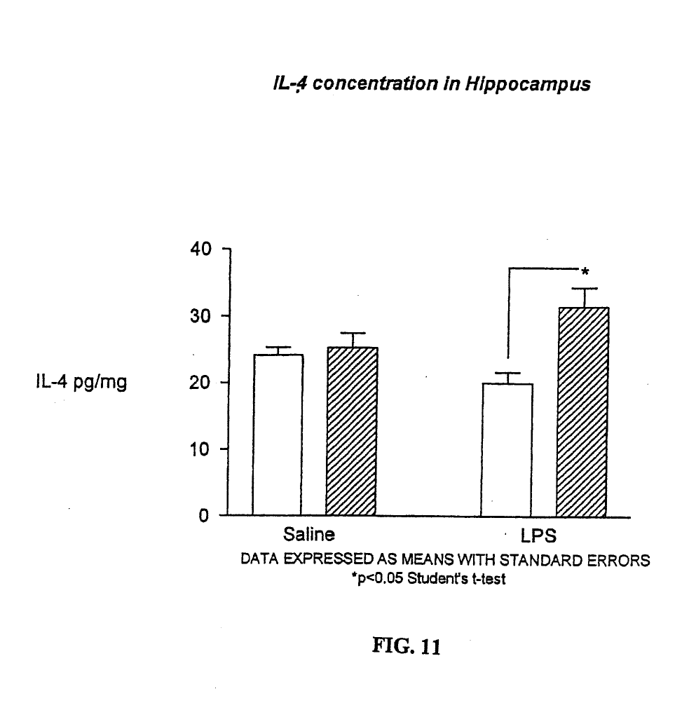

IL-4 is one of a number of cytokines secreted by the Th2 subclass of lymphocytes and is known for its anti-inflammatory effects. FIG. 11 shows that the IL-4 concentration in the hippocampus was significantly increased in the LPS group that had been pre-treated with the PG liposomes (*p<0.05). Open bars represent control group (Group E) and hashed bars represent the PG treated group (Group F). IL-4 was measured by ELISA and expressed as PG of IL-4 per mg of total protein. This upregulation of the anti-inflammatory cytokine IL-4 in the brain is indicative of the use of the process and composition of preferred embodiments of the present invention in treating a wide range of neuroinflammatory disorders, including Parkinson's disease, ALS, chronic inflammatory demyelinating disease CIPD and Guillain Barr syndrome.

### Example 11

IL-1β is one of a number of cytokines secreted by the Th1 subclass of lymphocytes and is known for its proinflammatory effects. Spleens from animals treated as described in Example 9, groups C and D thereof, were extracted and spleen cells collected. They were prepared as follows:

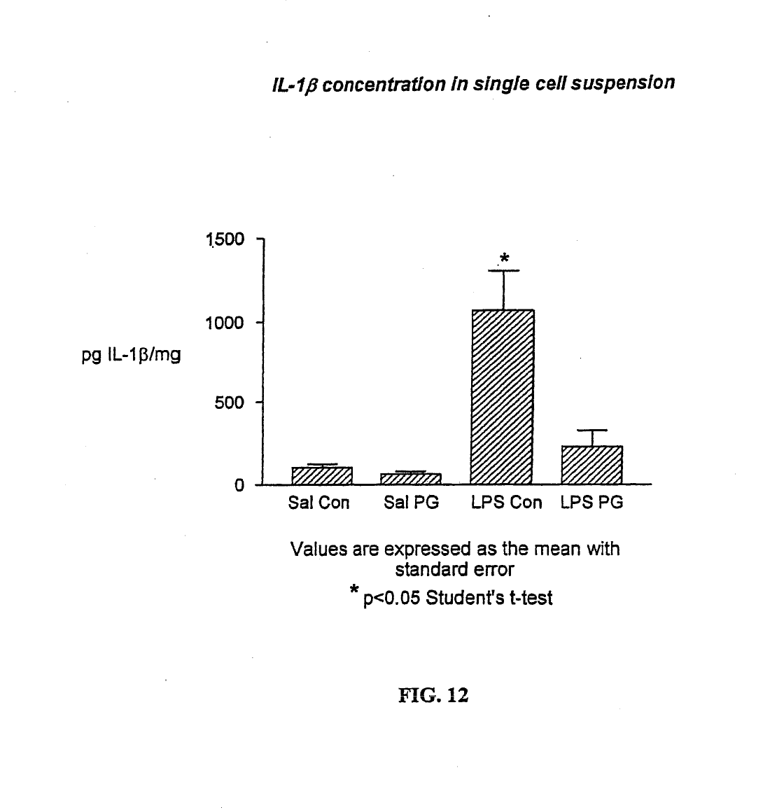

FIG. 12 shows that the IL-1β concentration in spleen cells was significantly reduced in the LPS group that had been pre-treated with the PG liposomes (*p<0.05). IL-1β was measured by ELISA and expressed as picagrams of IL-1β per mg of total protein. This indicates a systemic inflammatory effect of the process and compositions of preferred embodiments of the present invention.

### Example 12

U937 is a monocytic leukemia cell line that can be differentiated into macrophages by administration of phorbol esters. Treatment with lipopolysaccharide (LPS), a component of the cell wall of Gram-negative bacteria, stimulates an inflammatory response in U937 cells, with the upregulation of expression of a number of inflammatory molecules including TNFα. This model provides an experimental system for the assessment of anti-inflammatory therapies. The macrophages can be grown in culture medium in the presence of a suspected anti-inflammatory composition, and the expression of TNFα can be measured.

Liposomes of 100±20 nm in average diameter were prepared according to standard methods known in the art and had a composition of 75% phosphatidylglycerol (PG), 25% phosphatidylcholine (PC). The stock concentration of liposome was about 40 mM lipid and was diluted to the following final concentrations in the assay:

100 μM phosphatidylglycerol (PG)

### 40 μM PG

### 10 μM PG

### 4.0 μM PG

### 1 μM PG

The U937 cells were cultured by growing in RPMI medium (GIBCO BRL) supplemented with 10% fetal bovine serum (FBS) and 1% penicillin/streptomycin and grown at 37° C. in an atmosphere containing 5% CO₂. 5×10⁵ cells were seeded into wells of 6-well plates and caused to differentiated into macrophages by treatment with 150 nM phorbol myristate acetate (PMA) for 2-3 days. The cell medium was then replaced with complete medium after the U937 cells had differentiated into macrophages. The cells were then incubated for an additional 24 hrs to minimize pleotropic effects due to PMA treatment.

The cells were then incubated with either:

| | |
|---|---|
| Group A | Phosphate buffered saline (PBS) - as a negative control, |
| Group B | 10 ng/ml LPS - as a positive control, |
| Group C | 10 ng/ml LPS + 100 μM PG, |
| Group D | 10 ng/ml LPS + 40 μM PG, |
| Group E | 10 ng/ml LPS + 10 μM PG, |
| Group F | 10 ng/ml LPS + 4.0 μM PG, or |
| Group G | 10 ng/ml LPS + 1 μM PG. |

The cells were incubated as described above at 37° C. in 5% CO₂. After 18 hrs, the supernatants from each treatment were collected and assayed for TNF-α using a standard Quantikine Enzyme-Linked Immunosorbent Assay (ELISA) kit (R&D systems, Minneapolis, USA).

### Results

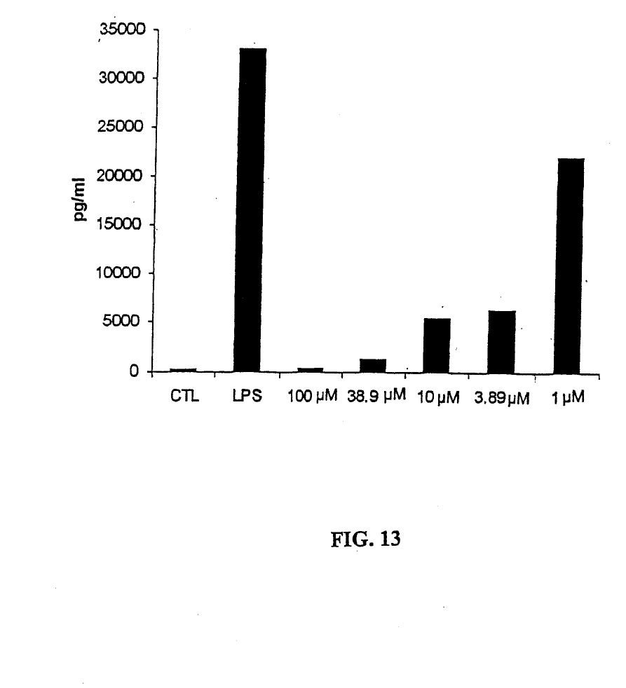

FIG. 13 shows the amount of secreted TNF-α in PG per ml. The results demonstrates that U937-differentiated macrophage cells express very low levels of TNF-α under normal conditions. However, once exposed to LPS, they secrete large amounts of TNF-α into the surrounding medium, which is indicative of cellular stress occurring. Incubation of the cells with PG liposomes inhibits the secretion of TNF-α in a dose-dependent manner, with the highest concentration of 100 μM resulting in a 98% decrease, and even the lowest concentration of 1 μM causing a 58% decrease in TNF-α expression.

### Example 13

To determine the effect of the PG liposomes of the preferred embodiment of the present invention on endothelial function, the endothelin-1 (ET-1) content in the ears of mice which had been subjected to the CHS studies as described in Example 3 was determined. Endothelin-1 is a potent vasoconstrictive agent, has inotropic and mitogenic actions, modulates salt and water homeostasis and plays an important role in the maintenance of vascular tone and blood pressure. Various lines of evidence indicate that endogenous ET-1 may contribute to the pathophysiology of conditions associated with sustained vasoconstriction, such as heart failure. In heart failure, elevated levels of circulating ET-1 and big-ET-1 are observed (Giannessi D, Del Ry S, Vitale R L. “The role of endothelins and their receptors in heart failure.” Pharmacol Res 2001 February 43:2 111-26). Thus ET-1 is a marker of endothelial function and increased production of ET-1 in tissue is indicative of impaired endothelial function.

In order to determine ET-1 expression, mouse ears (right challenged ear) were harvested 24 hrs after challenge in CHS experiments. Ears were obtained from mice injected intramuscularly with PBS for 6 days (Group A) and mice injected intramuscularly with 75% PG/25% PC liposomes (600,000 liposomes/injection; Group B). Ears were stored in RNA later at −20° C. until RNA extraction. RNA was extracted and cDNA was generated using reverse transcriptase (RT) along with ET-1-specific primers, as an internal control, PCR was also performed using β-actin-specific primers. PCR products were resolved on a 1.5% agarose gel and the DNA bands were quantitated by densitometry analysis. The ratio of ET-1/β-actin was calculated.

PCR Preparation:

| PCR Mix (ET-1) | PCR Mix (β-Actin) |
|---|---|
| 5 μl PCR Buffer (10×) | 5 μl PCR Buffer (10×) |
| 1.5 μl MgCl2 (50 mM) | 1.5 μl MgCl2 (50 mM) |
| 1 μl dNTP (10 mM) | 1 μl dNTP (10 mM) |
| 0.5 μl Primer 1 (25 uM) | 1 μl Primer 1 (10 uM) |
| 0.5 μl Primer 2 (25 uM) | 1 μl Primer 2 (10 uM) |
| 0.25 μl TAQ | 0.25 μl TAQ |
| 2.5 μl cDNA | 2.5 μl cDNA |
| 38 μl Water | 37.75 μl Water |
| 50 μl Total | 50 μl Total |

Primers: (as previously described in Yang, et al. “Conditional cardiac overexpression of endothelin-1 in transgenic mice,” FASEB J. 15(5): A1138-A1138 Part 2 (2001)).

ET-1(r)
5′-CAG CAC TTC TTG TCT TTT TGG-3′
ET-1(f)
5′-CCA AGG AGC TCC AGA AAC AG-3′
β-Actin(F)
5′-GTG GGC CGC TCT AGG CAC CAA-3′
β-Actin(r)
5′-CTC TTT GAT GTC ACG CAC GAT TTC-3′

PCR Settings:

94° C.—5 minutes

[symbol] 94° C.—30 s

60° C.—30 s 30 cycles

72° C.—60 s

72° C.—10 minutes

4° C.—Soak

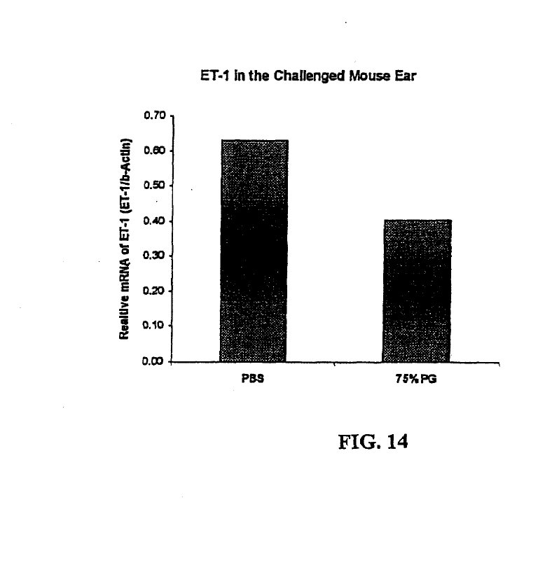

After 6-daily injections of the 75% PG liposomes, the level of ET-1 was decreased by 36% relative to control mice receiving PBS during the same injection regimen. The results are shown graphically on FIG. 14. This decrease indicates a beneficial effect resulting from the injection of the liposomes of the preferred embodiment of the invention on endothelial function in a mammalian patient, through Th1 mediated inflammation reduction.

### Example 14

Intercellular adhesion molecule-1 (ICAM-1) is a cell surface molecule expressed by several cell types, including leukocytes and endothelial cells. It is involved in the adhesion of monocytes to endothelial cells and plays a role in inflammatory processes and in the T-cell mediated host defense system. ICAM-1 expression probably contributes to the clinical manifestations of a variety of diseases, predominantly by interfering with normal immune function. Among these are malignancies (e.g., melanoma and lymphomas), many inflammatory disorders (e.g., asthma and autoimmune disorders), atherosclerosis, ischemia, certain neurological disorders, and allogeneic organ transplantation (Van de Stolpe A, van der Saag P T, “Intercellular adhesion molecule-1” J. Mol. Med. (1996) 74:113-33).

Human umbilical vein endothelial cells (HUvECs) are a primary cell line of endothelial cells that are isolated from umbilical vein cords as follows.

T75 flasks were prepared by coating with 0.2% gelatin (5-7 ml/flask) for a minimum of 15/20 minutes or overnight. The excess was then removed. The cord was sprayed with 70% ethanol prior to procedure and any placenta still remaining attached to the cord was cut away. The cord was then cut to an approximate length of 5-6 inches. The cord has two arteries which are thick walled and one vein that is bigger and thin walled. The vein was located and the serrated edge of a stopper placed into it. Approximately 20 cm of string was then used to tie the cord onto the stopper.

The cord was then washed through with phosphate buffered saline (PBS) a number of times until the PBS ran clear. Following this 15-20 mls of Collagenase solution was placed into the cord; it was wrapped in tinfoil and incubated for 15 minutes at 37° C. After incubation the tied end of the cord was cut and the collagenase drained into a 50 ml tube. Collagenase was then passed through the cord again, the cord was massaged to loosen the endothelial cells and then PBS was passed through the cord and collected into the same tube containing the collagenase solution. This was then centrifuged at 1600 RPMs, the supernatant removed and the pellet resuspended in 10-12 mls of M199 complete medium. Finally the medium containing the cells was added to the gelatinized flasks.

Liposomes of 100±20 nm in average diameter were prepared according to standard methods known in the art and had a composition of 75% phosphatidylglycerol (PG), 25% phosphatidylcholine (PC). The stock concentration of liposome was 40 mM lipid and was diluted to 100 μM in the assay.

HUVECs split into a number of tissue culture flasks, allowed to adhere to the surface of the flask and then treated as follows:

Group A —PBS—as a negative control,
Group B—500 ng/ml LPS—as a positive control,
Group C—500 ng/ml LPS+100 μM PG
Group D—500 ng/ml LPS+100 μM PC

The cells were incubated at 37° C., 5% CO₂. After 18 hrs, the supernatants from each treatment were collected and assayed for ET-1 using a standard ELISA kit (obtained from Assay Designs) and the cells harvested for analyzing ICAM-1 as follows.

The cells were first washed with PBS and then incubated with a cell dissociation buffer at 37° C. for 25-30 min. The cells were then washed by centrifugation and incubated with an anti-CD54 (ICAM-1) antibody for 30 minutes. A secondary FITC antibody was then added and incubated with the cells as before. Finally they were resuspended in 1 ml of PBS and analyzed for fluorescence on a flow cytometer.

### Results

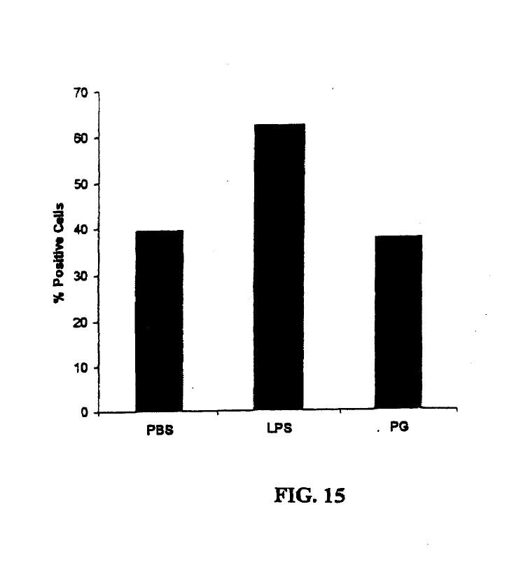

The results are presented on FIG. 15, a graphical presentation of the percentage of cells staining positive for ICAM-1 in the respective cultures. It is to be noted that the numbers of cells staining positive in the PG liposome-containing culture is reduced to negative control level, and is much lower than the positive control level.

### Example 15

Microglial cells (brain macrophages) were cultured, and their output of TNF-α, an inflammatory cytokine, was measured. The cells were stimulated with the immunoglobulin (IgG) of patients suffering from ALS, and the TNF-α output increased about 800-fold as a result. When the same cells were grown in the presence of both the ALS IgG and PG liposomes, output of TNF-α decreased by about 75%, indicating the potential of the preferred embodiments of the present invention in the treatment of ALS.

1-12. (canceled)

13. A method for treating a T-cell function-mediated disorder comprising administering to a mammalian patient an effective amount of pharmaceutically acceptable bodies comprising an effective number of phosphate-glycerol groups to inhibit and/or reduce the progression of the T-cell function-mediated disorder.

14. The method according to claim 13, wherein said bodies are liposomes.

15. The method according to claim 14, wherein said liposomes have a size from about 20-1000 nm.

16. The method according to claim 15, wherein said phosphate-glycerol groups comprise from about 60 to 100% of groups on said bodies.

17. The method according to claim 16, wherein said phosphate-glycerol groups comprise about 75% of groups on said bodies.

18-32. (canceled)

33. A method for treating a T-cell function-mediated disorder comprising administering to a mammalian patient suffering from or at risk of suffering from a T-cell function mediated disorder, an effective amount of a composition comprising pharmaceutically acceptable bodies having a size from about 20 nm to about 500 μm, comprising on the surface thereof a plurality of phosphate-glycerol groups, or groups convertible to said phosphate-glycerol groups, such that upon administration, the progression of the T-cell function mediated disorder is inhibited and/or reduced.

34. The method according to claim 33, wherein said bodies are liposomes.

35. The method according to claim 34, where in said liposomes have a size from about 20-1000 nm.

36. The method according to claim 35, wherein said phosphate-glycerol groups comprise from about 60 to 100% of groups on said bodies.

37. The method according to claim 36, wherein said phosphate-glycerol groups comprise about 75% of groups on said bodies.

38-57. (canceled)

58. The method as in any of claims 13-17 or 33-37, wherein said bodies are essentially free of non-lipid pharmaceutically acceptable entities.

59. The method as in any of claims 13-17, or 33-37, wherein said bodies are free of non-lipid pharmaceutically acceptable entities.

60. The method as in any of claims 16, or 36, wherein remaining groups comprise phosphate-choline.

61. The method as in any of claims 17, or 37, wherein remaining groups comprise phosphate-choline.

62-73. (canceled)


SEQUENCE LISTING (4 sequences)

SEQ ID NO: 1 - DNA, 21 bp, Artificial Sequence
  Description of Artificial Sequence Synthetic primer
cagcacttcttgtctttttgg

SEQ ID NO: 2 - DNA, 20 bp, Artificial Sequence
  Description of Artificial Sequence Synthetic primer
ccaaggagctccagaaacag

SEQ ID NO: 3 - DNA, 21 bp, Artificial Sequence
  Description of Artificial Sequence Synthetic primer
gtgggccgctctaggcaccaa

SEQ ID NO: 4 - DNA, 24 bp, Artificial Sequence
  Description of Artificial Sequence Synthetic primer
ctctttgatgtcacgcacgatttc